(19) United States
(12) Patent Application Publication
(10) Pub. No.: US 2006/0224245 A1
(43) Pub. Date: Oct. 5, 2006

(54) Hip prosthesis

(76) Inventor: Thomas Siebel, Saarbrucken (DE)

Correspondence Address: FRIEDRICH KUEFFNER, 317 MADISON AVENUE, SUITE 910, NEW YORK, NY, 10017, US

(21) Appl. No.: 10/553,071

(22) PCT Filed: Apr. 7, 2004
(86) PCT No.: PCT/EP04/03701

§ 371 Date: Oct. 13, 2005

(30) Foreign Application Priority Data
Apr. 15, 2003 (DE) 103177663

(51) Int. Cl. A61F 2/32

(52) U.S. Cl. 623/22.11

(57) ABSTRACT

The invention relates to a hip prosthesis comprising a joint part (6) and an anchoring part (5) to be implanted in the neck (2) of the femur (1). According to the invention, the anchoring part (5) can be deformed in a manner that corresponds with load-associated deformations of the femur (1) in the area of the neck (2). The anchoring part is preferably formed from a number of parts consisting of parts (9, 10), which can move relative to one another and which are positively interconnected in a direction perpendicular to the longitudinal axis (4) of the femoral neck.

The invention concerns a hip prosthesis with a joint part and an anchoring part to be implanted in the neck of the femur.

Hip prostheses of this type are known from experience, whose anchoring part is formed by a conical body, from which a coaxial pin extends distally. The anchoring part is inserted into the cut-off neck of the femur in the direction of the longitudinal axis of the neck of the femur, such that the pin emerges laterally from the femur and is fastened at the point of emergence by a mounting plate. The conical body has a flange-like collar that rests, first of all, against the compact tissue at the cut surface.

A disadvantage of this is that the mounting plate causes discomfort due to muscles and nerves that pass close to it. Due to disintegration of the bone tissue, the flange-like collar can lose its support function, so that the mounting plate pain increases. Further bone disintegration finally leads to failure of the prosthesis.

The objective of the present invention is to develop a new hip prosthesis, which is to be implanted only in the neck of the femur and continues to function, largely without patient discomfort, over a longer period of time than previously known hip prostheses of this type.

This objective is achieved by the hip prosthesis of the invention, which is characterized by the fact that the anchoring part can be deformed in a manner that corresponds to load-induced deformations of the femur in the area of the neck. A hip prosthesis in accordance with the invention, on the one hand, takes on the necessary bearing functions and, on the other hand, allows, due to suitable elasticity, small deformations in the neck region of the femur, which already occur under normal loads, even in healthy bone. The bone remains sufficiently loaded. Inactivity atrophy is avoided.

Under these conditions, additional fastening of the anchoring part laterally on the femur can be dispensed with, and in accordance with a preferred embodiment of the invention, the anchoring part can be designed as a stump that freely terminates distally in the bone tissue. Mounting plate pain does not occur. The prosthesis causes no symptoms during prolonged use. Naturally, the stump must have a sufficient lever length to perform its support function.

In a preferred embodiment of the invention, the anchoring part is formed by a number of parts that can move relative to one another and are positively joined with one another in the direction perpendicular to the longitudinal axis of the neck of the femur, such that the parts can be displaced and/or rotated relative to one another. This allows the anchoring part to adjust both to small compressive forces and small bending forces. Finally, adjustments to torsion of the neck of the femur is also possible.

In a preferred embodiment of the invention, a first part consists of an anchoring block and a guide stem that projects from the anchoring block in the direction of the longitudinal axis of the neck of the femur, and a second part consists of a guide block with a guide channel that receives the guide stem.

The guide stem and guide channel can have corresponding cross sections, so that the second part can be displaced on the guide stem in the direction of the longitudinal axis of the neck of the femur and possibly rotated on the guide stem about the longitudinal axis of the neck of the femur.

However, the guide channel is preferably expanded, so that the second part can be rotated relative to the first part about an axis that is perpendicular to the longitudinal axis of the neck of the femur.

Preferably, projections or lugs located opposite each other are formed in the guide channel. The second part can be supported and rotated on these projections or lugs like a rocker. In the case of rectangular cross sections of the guide channel and guide stem, e.g., rotation of the second part in a certain plane that intersects the longitudinal axis of the neck would be possible on opposing projections of this type.

In another embodiment of the invention, the cross sections of the guide stem and guide channel are circular, and an annular projection is formed in the guide channel. In this embodiment, rotation of the second part is possible in each plane that intersects the longitudinal axis of the neck of the femur.

It is advantageous for the opposing projections to have rolling surfaces, so that the projections rest against the guide stem with no play or approximately no play in each rotational position of the second part. Undesired translational movements of the second part perpendicular to the longitudinal axis of the guide stem are avoided in this way.

In another advantageous refinement of the invention, an annular recess, into which bone tissue can grow, can be formed between the anchoring blocks of the parts. The bone tissue that grows into the annular space provides for stable anchoring of the prosthesis in the longitudinal direction. Advantageously, the movable anchoring block of the second part also provides for loading of this bone tissue, and this loading promotes both the formation and preservation of the bone tissue.

In another refinement of the invention, fins can project from the anchoring part, preferably radially with respect to the longitudinal axis of the neck of the femur. The fins increase the contact surface between the anchoring part and the bone tissue and thus the stability of the anchoring.

The invention will now be explained in greater detail with reference to specific embodiments and the accompanying drawings that illustrate these embodiments.

FIG. 1 shows a prosthesis of the invention implanted in the neck of the femur.

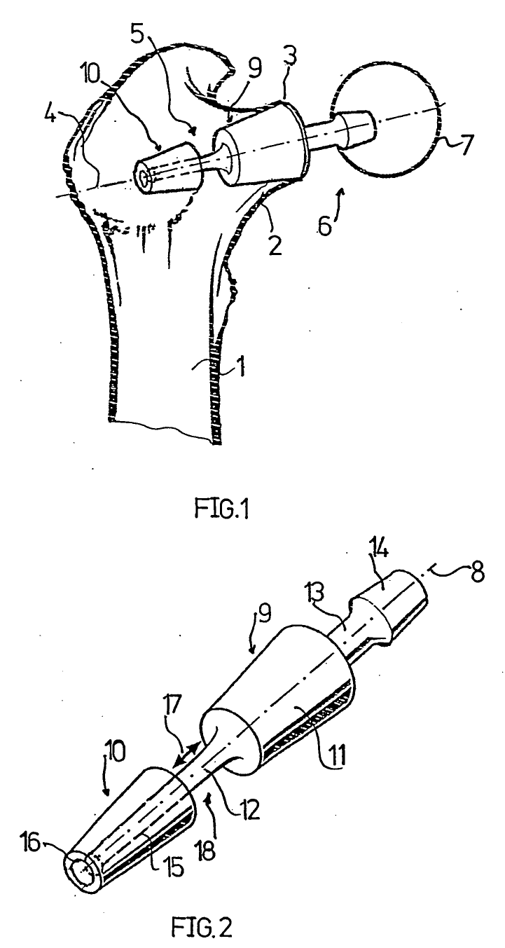

FIG. 2 shows a portion of the prosthesis shown in FIG. 1.

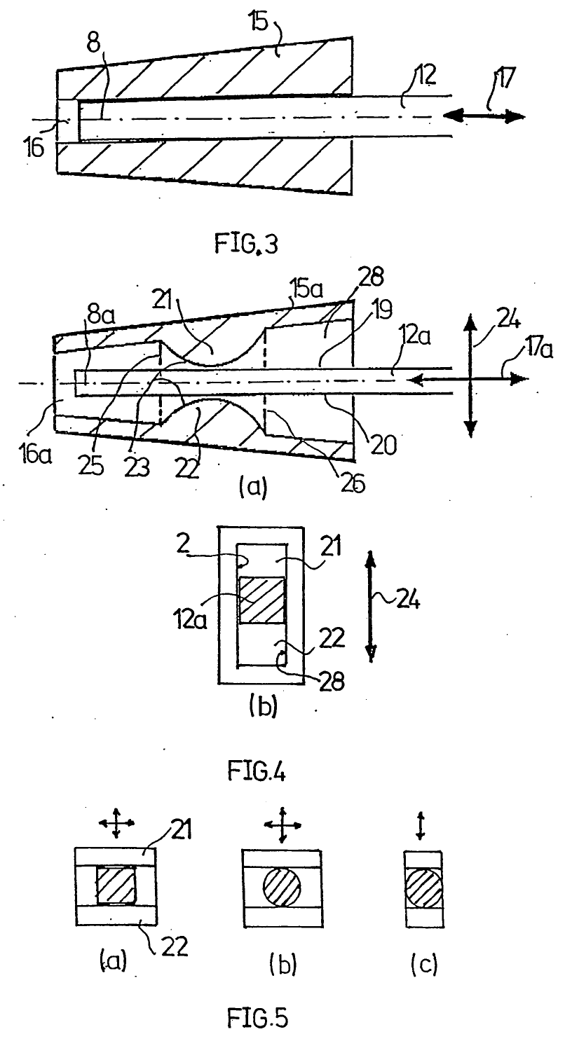

FIG. 3 shows an anchoring block of the prosthesis in FIG. 1, which can be moved on a guide stem.

FIG. 4 shows a longitudinal section and a cross section of another embodiment of an anchoring block that can be moved on a guide stem.

FIG. 5 shows possible variations of the embodiment of FIG. 4.

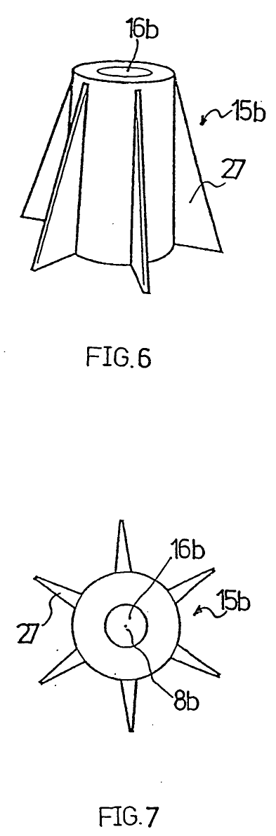

FIG. 6 shows a side view of an anchoring block that is provided with fins.

FIG. 7 shows the anchoring block of FIG. 5 in a view from below.

FIG. 1 shows a femur 1, whose neck 2 is cut off at 3 perpendicular to the longitudinal axis 4 of the neck of the femur.

A prosthesis, which has an anchoring part 5 embedded in the bone tissue and a joint part 6 with a joint ball 7 that projects from the cutting plane at 3, is implanted in the neck of the femur 1.

The anchoring part 5 of the prosthesis, which is rotationally symmetric with respect to a longitudinal axis 8, consists of parts 9 and 10, which can be moved relative to each other. Part 9 has a conical anchoring block 11, from whose distal end a guide stem 12 extends. An extension 13, which belongs to the joint part and has a cone 14 for connection with the joint ball 7, extends from the proximal end of the anchoring block 11.

Part 10 of the anchoring part 5 consists of an anchoring block 15 with a guide channel 16, which receives the guide stem 12 of part 9. A double arrow 17 indicates that the guide stem 12 can be moved back and forth in the guide channel 16 in the direction of the longitudinal axis 8.

As FIGS. 1 and 2 also show, an annular recess 18 is formed between the anchoring blocks 11 and 15 of the parts 9 and 10.

In a certain position of displacement of the parts 9 and 10, the surface lines of the two conical anchoring blocks 11, 15 form a continuous straight line.

As FIG. 3 shows, the cross section of the guide stem 12 and the guide channel 16 are about the same size in this embodiment, so that there is no clearance perpendicular to the longitudinal axis 8, and the anchoring block 15 can move back and forth only in the direction of the double arrow.

If the guide stem and the guide channel have circular cross sections, the anchoring block 15 can make not only the translational movement indicated by the double arrow 17, but also a rotational movement about the longitudinal axis 8. If this possibility of rotation is not desired, the cross sections can have a shape different from a circular shape, e.g., a square shape.

In a departure from these embodiments, the cross-sectional area of the guide stem 12 could also be smaller than the cross-sectional area of the guide channel 16, so that in addition to translational movements in the direction of the longitudinal axis 8, translational movements perpendicular to the longitudinal axis 8 and rotational movements relative to the guide stem 12 and thus to the anchoring block 11 about an axis perpendicular to the longitudinal axis 8 are also possible.

The embodiment in FIG. 4 shows an anchoring block 15a with a guide channel 16a, whose cross-sectional area is smaller than the cross-sectional area of a guide stem 12a held in the guide channel. The guide channel 16a and the guide stem 12a have a rectangular cross section, and lateral surfaces of the guide stem 12a, which are parallel to the plane of the drawing in FIG. 4a, rest against corresponding inner walls 27 and 28 of the guide channel 16a. Projections or lugs 21 and 22 face other lateral surfaces 19 and 20 of the guide stem 12a. They project from the walls of the guide channel 16a opposite the lateral surfaces 19 and 20 and are arranged opposite one another. The projections 21 and 22 allow the anchoring block 15a to move like a rocker on the guide stem 12a. Each of the projections has a round rolling surface 23, so that when the anchoring part 15a swivels about an axis perpendicular to the plane of the drawing according to arrow 24, the projections always remain in contact with the guide stem 12a with little or no play.

In a departure from the embodiment shown in FIG. 4, the guide channel 16a and the guide stem 12a can also be provided with circular cross sections, and an annular projection can be formed in the guide channel, as indicated in FIG. 4 by the broken lines 25 and 26. In this case, the anchoring block can turn on the guide stem like a rocker about any plane that intersects the longitudinal axis 8a.

Other possible variations of the embodiment in FIG. 4 are shown in FIGS. 5a to 5c as cross-sectional views.

In FIG. 5a, in contrast to the embodiment shown in FIG. 4, the lateral surfaces of the guide stem that are parallel to the plane of the drawing do not rest against the inner walls of the guide channel that are located opposite them. There is thus the additional possibility of a translational movement of the guide stem along the crests of the projections 21 and 22 and of rotation about an axis that perpendicularly intersects the crests of the projections 21 and 22.

In the embodiment shown in FIG. 5b, a guide stem is selected with a circular cross section, so that in addition to the possible movements according to FIG. 4 and FIG. 5a, rotation can also occur on the longitudinal axis of the guide stem.

According to FIG. 5c, a guide stem with a circular cross section rests against both projections as well as against the sidewalls perpendicular to the crests of the projections. Besides a rotation about the longitudinal axis of the guide stem in accordance with FIG. 5b, a rotation of the guide stem about an axis parallel to the plane of the drawing is possible.

Reference is now made to FIGS. 6 and 7, which show an anchoring block 15b that has been modified by fins 27 and has a guide channel 16b. The triangular fins project from the block radially with respect to the longitudinal axis 8b. The fins 27 increase the contact surface with the surrounding bone tissue and increase the stability of the anchoring.

To implant the prosthesis described above, a channel coaxial with the longitudinal axis 4 of the neck of the femur, which has been cut off at 3, is milled into the neck of the femur perpendicular to the cutting plane of the neck of the femur. The channel has approximately the same shape as the conical anchoring part 5 but is narrower, so that the parts 9 and 10 can be driven into the bone tissue and frictionally connected.

The implant grows into and together with the bone tissue, and especially the annular recess 18 becomes filled by bone tissue growing into it.

When the bone or the hip joint is subjected to a load, the length of the anchoring part 5 is sufficient to apply the necessary leverage. Moreover, load-induced deformations of the bone in the neck region, as would also occur in healthy bone, are not blocked by the implant, which itself is able to move, so that the anchoring part can adapt, and bending movements can occur. In addition, adjustment to torsion of the neck of the femur about its longitudinal axis is possible in itself.

1. Hip prosthesis with a joint part (6) and an anchoring part (5) to be implanted in the neck (2) of the femur (1), wherein the anchoring part (5) can be deformed in a manner that corresponds to load-induced deformations of the femur (1) in the area of the neck (2).

2. Hip prosthesis in accordance with claim 1, wherein the anchoring part (5) is designed as a stump that freely terminates distally in the bone tissue.

3. Hip prosthesis in accordance with claim 1, wherein the anchoring part (5) is formed by several parts (9, 10) that can move relative to one another and are positively joined with one another in the direction perpendicular to the longitudinal axis (4) of the neck of the femur.

4. Hip prosthesis in accordance with claim 3, wherein the parts (9, 10) can be displaced and/or rotated relative to one another.

5. Hip prosthesis in accordance with claim 3, wherein a first part (9) consists of an anchoring block (11) and a guide stem (12) that projects from the anchoring block (11) in the direction of the longitudinal axis (4) of the neck of the femur, and a second part (10) consists of a guide block (15) with a guide channel (16) that receives the guide stem (12).

6. Hip prosthesis in accordance with claim 5, wherein the guide channel (16a) is expanded relative to the guide stem (12a), and the second part (10) can be rotated relative to the first part (9) about an axis that is perpendicular to the longitudinal axis (4) of the neck of the femur.

7. Hip prosthesis in accordance with claim 5, wherein projections (21, 22) located opposite each other are formed in the guide channel (16a), and that the first part can rotate on these projections like a rocker relative to the second part.

8. Hip prosthesis in accordance with claim 5, wherein the cross sections of the guide stem and guide channel are circular, and an annular projection (25, 26) is formed in the channel.

9. Hip prosthesis in accordance with claim 8, wherein the opposing projections (21, 22) have rounded rolling surfaces (23), so that the projections (21, 22) rest against the guide stem (12a) in each rotational position of the second part.

10. Hip prosthesis in accordance with claim 1, wherein an annular recess (18), which can become filled by bone tissue growing into it, is formed between the anchoring blocks (11, 15) of the parts (9, 10).
